(19) United States
(12) Patent Application Publication
(10) Pub. No.: US 2006/0246018 A1
(43) Pub. Date: Nov. 2, 2006

(54) Mixture consisting of uv-a and uv-b filters

(76) Inventors: Valerie Andre, Ludwigshafen (DE); Thomas Wunsch, Speyer (DE); Thomas Heidenfelder, Dannstadt (DE)

Correspondence Address: CONNOLLY BOVE LODGE & HUTZ, LLP, P O BOX 2207, WILMINGTON, DE, 19899, US

(21) Appl. No.: 10/561,001

(22) PCT Filed: Jun. 8, 2004
(86) PCT No.: PCT/EP04/06158

§ 371 Date: Dec. 16, 2005

(30) Foreign Application Priority Data
Jun. 24, 2003 (DE) 10328547.4

(51) Int. Cl. A61K 8/37

(52) U.S. Cl. 424/59

(57) ABSTRACT

The invention relates to a mixture consisting of a UV-A filter of the formula I and a UV-B filter of the formula II.

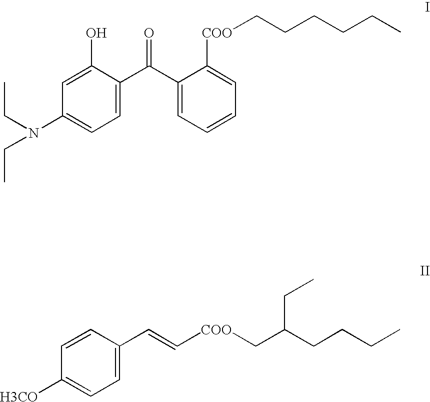

The present invention relates to a mixture consisting of a UV-A filter of the formula I and a UV-B filter of the formula II.

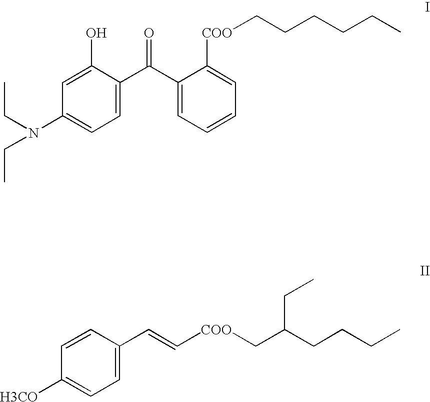

The use of amino-substituted hydroxybenzophenones as photostable UV filters in cosmetic and dermatological preparations was described for the first time in EP 1 046 391 A2.

Cosmetic preparations comprising mixtures of amino-substituted hydroxybenzophenones with compounds which absorb in the UV-A region, in the UV-B region or over both regions are described, inter alia, in EP 1 133 980 A2, EP 1 240 984 A2, EP 1 291 009 and in WO 03/039507.

An amino-substituted hydroxybenzophenone used preferably for sunscreen compositions is the compound of the formula I mentioned at the start, which is manufactured and sold under the trade name Uvinul® A Plus by BASF.

The preparation and purification of Uvinul® A Plus, described in DE-A-10221805, takes place, inter alia, by treatment with an adsorbent and subsequent distillative removal of the solvent. The end product obtained in this way can be packaged following distillation as melt.

In this connection, it can happen that the ester crystallizes out of the melt during storage and the product can only be removed from the pack by repeated melting. For many manufacturers of cosmetic preparations who do not have the required apparatuses for melting solid substances, this presents considerable applications-related problems.

It was therefore an object of the present invention to provide Uvinul® A Plus in a form with which undesired crystallizing out of this UV-A filter from its melt is prevented.

This object was achieved through the provision of a mixture consisting of a UV-A filter of the formula I and a UV-B filter of the formula II.

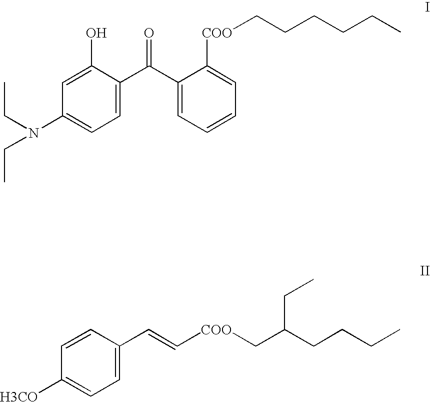

2-Ethylhexyl p-methoxycinnamate of the formula II is a colorless oil and is one of the UV-B filters most often used in cosmetic and dermatological preparations. Compound II is obtainable from BASF under the name Uvinul® MC 80.

For the manufacturer of photoprotective agent preparations, the mixture according to the invention offers the advantage that undesired crystallizing out of Uvinul® A Plus, in particular in the packaging packs, is prevented and the problems associated with this upon emptying these packs no longer arise. It is thus no longer necessary to store packs containing Uvinul® A Plus, for example bottles or containers, at temperatures >20° C.

In addition, the mixture according to the invention constitutes a photostable broadband filter which makes it possible to absorb both UV-A rays and also UV-B rays.

A preferred embodiment of the abovementioned mixture is one which consists of 30 to 70% by weight, particularly preferably 30 to 50% by weight, very particularly preferably 35 to 45% by weight, of the UV. A filter I and 70 to 30% by weight, particularly preferably 70 to 50% by weight, very particularly preferably 65 to 55% by weight, of the UV-B filter 11.

A further preferred embodiment of the mixture is one which is present in liquid form with excellent flow behavior.

The viscosities of the mixtures according to the invention are in the range from 100 to 800 mPa*s, preferably in the range from 200 to 600 mPa*s, particularly preferably from 300 to 500 mPa*s.

This mixture is prepared in a manner known per se by simply mixing the compounds I and II, the order of the addition of the individual components being of no importance.

The present invention also provides the use of a mixture defined according to one of claims 1 to 3 for producing cosmetic and pharmaceutical preparations for protecting human skin and human hair against UV radiation.

The cosmetic and pharmaceutical preparations comprising photoprotective agents are generally based on a carrier which comprises at least one oil phase. However, preparations solely based on water are also possible when using compounds with hydrophilic substituents. Accordingly, oils, oil-in-water and water-in-oil emulsions, creams and pastes, lip protection stick masses or grease-free gels are suitable.

Suitable emulsions are, inter alia, also O/W macroemulsions, O/W microemulsions, W/O/W emulsions or O/W/O emulsions with amino-substituted hydroxybenzophenones of the formula I present in dispersed form, the emulsions being obtainable by phase-inversion technology, as in DE-A-197 26 121.

Customary cosmetic auxiliaries which may be suitable as additives are, for example, coemulsifiers, fats and waxes, stabilizers, thickeners, biogenic active ingredients, film formers, fragrances, dyes, pearlizing agents, preservatives, pigments, electrolytes (e.g. magnesium sulfate) and pH regulators. Suitable coemulsifiers are preferably known W/O emulsifiers and also O/W emulsifiers, such as, for example, polyglycerol esters, sorbitan esters or partially esterified glycerides. Typical examples of fats are glycerides; waxes to be mentioned are, inter alia, beeswax, paraffin wax or microwaxes, if appropriate in combination with hydrophilic waxes. Stabilizers which can be used are metal salts of fatty acids, such as, for example, magnesium stearate, aluminum stearate and/or zinc stearate. Suitable thickeners are, for example, crosslinked polyacrylic acids and derivatives thereof, polysaccharides, in particular xanthan gum, guar guar, agar agar, alginates and tyloses, carboxymethylcellulose and hydroxyethyl-cellulose, also fatty alcohols, monoglycerides and fatty acids, polyacrylates, polyvinyl alcohol and polyvinylpyrrolidone. Biogenic active ingredients are understood as meaning, for example, plant extracts, protein hydrolysates and vitamin complexes. Customary film formers are, for example, hydrocolloids, such as chitosan, microcrystalline chitosan or quaternized chitosan, polyvinylpyrrolidone, vinylpyrrolidone-vinyl acetate copolymers, polymers of the acrylic acid series, quaternary cellulose derivatives and similar compounds. Suitable preservatives are, for example, formaldehyde solution, p-hydroxybenzoate or sorbic acid. Suitable pearlizing agents are, for example, glycol distearic esters, such as ethylene glycol distearate, but also fatty acids and fatty acid monoglycol esters. Dyes which can be used are the substances approved and suitable for cosmetic purposes, as are listed, for example, in the publication “Kosmetische Fätrbemittel” [“Cosmetic Colorants”] from the Farbstoff-kommission der Deutschen Forschungsgemeinschaft [Dyes Commission of the German Research Society], published in Verlag Chemie, Weinheim, 1984. These dyes are usually used in concentration of from 0.001 to 0.1% by weight, based on the total mixture.

An additional content of antioxidants is generally preferred. Favorable antioxidants which may be used are all antioxidants which are customary or suitable for cosmetic and/or dermatological applications.

Advantageously, the antioxidants are chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximines, buthionine sulfones, penta-, hexa-, heptathionine sulfoximine) in very low tolerated doses (e.g. pmol to pmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, biliburin, biliverdin, EDTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives thereof (e.g. ascorbyl palmitate, Mg ascorbyl phosphate, ascorbyl acetate), tocopherol and derivatives (e.g. vitamin E acetate, tocotrienol), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

The amount of the abovementioned antioxidants (one or more compounds) in the preparations is preferably 0.001 to 30% by weight, particularly preferably 0.05 to 20% by weight, in particular 1 to 10% by weight, based on the total weight of the preparation.

If vitamin E and/or derivatives thereof are the antioxidant or the antioxidants, it is advantageous to choose their particular concentration from the range from 0.001 to 10% by weight, based on the total weight of the formulation.

If vitamin A and/or derivatives thereof or carotenoids are the antioxidant or the antioxidants, it is advantageous to choose their particular concentration from the range from 0.001 to 10% by weight, based on the total weight of the formulation.

Customary oil components in cosmetics are, for example, paraffin oil, glyceryl stearate, isopropyl myristate, diisopropyl adipate, cetylstearyl 2-ethylhexanoate, hydrogenated polyisobutene, Vaseline, caprylic/capric triglycerides, microcrystalline wax, lanolin and stearic acid.

The total fraction of auxiliaries and additives can be 1 to 80% by weight, preferably 6 to 40% by weight and the nonaqueous fraction (“active substance”) can be 20 to 80% by weight, preferably 30 to 70% by weight—based on the compositions. The compositions can be prepared in a manner known per se, i.e. for example by hot, cold, hot-hot/cold or PIT emulsification.

Such sunscreen preparations can, accordingly, be in liquid, paste or solid form, for example as water-in-oil creams, oil-in-water creams and lotions, aerosol foam creams, gels, oils, fatty sticks, powders, sprays or alcoholic-aqueous lotions. Finally, further substances which absorb in the UV region and are known per se can be co-used if they are stable in the overall system of the combination of UV filters to be used according to the invention.

The majority of photoprotective agents in the cosmetic and pharmaceutical preparations which serve to protect the human epidermis consists of compounds which absorb UV light in the UV-B region, i.e. in the range from 280 to 320 nm. For example, the fraction of UV-A absorbers to be used according to the invention is 10 to 90% by weight, preferably 20 to 50% by weight, based on the total amount of UV-B and UV-A absorbing substances.

Suitable UV filter substances which are used in combination with the mixture to be used according to the invention are any UV-A and UV-B filter substances. Examples thereof are:

| | | CAS No. |
|---|---|---|
| No. | Substance | (≃acid) |
| 1 | 4-Aminobenzoic acid | 150-13-0 |
| 2 | 3-(4′Trimethylammonium)benzylidenebornan-2-one | 52793-97-2 |
| | methyl sulfate | |
| 3 | 3,3,5-Trimethylcyclohexyl salicylate (homosalate) | 118-56-9 |
| 4 | 2-Hydroxy-4-methoxybenzophenone (oxybenzone) | 131-57-7 |
| 5 | 2-Phenylbenzimidazole-5-sulfonic acid and its | 27503-81-7 |
| | potassium, sodium and triethanolamine salts | |
| 6 | 3,3′-(1,4-Phenylenedimethine)bis(7,7-dimethyl- | 90457-82-2 |
| | 2-oxobicyclo-[2.2.1]heptane-1-methanesulfonic | |
| | acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Hydroxy-4-methoxybenzophenone-5- | 4065-45-6 |
| | sulfone(sulisobenzone) and the sodium salt | |
| 12 | 3-(4′-Sulfo)benzylidenebornan-2-one and salts | 58030-58-6 |
| 13 | 3-Benzylidenebornan-2-one | 16087-24-8 |
| 14 | 1-(4′-Isopropylphenyl)-3-phenylpropane-1,3-dione | 63260-25-9 |
| 15 | 4-Isopropylbenzyl salicylate | 94134-93-7 |
| 16 | 2,4,6-Trianiline(o-carbo-2′-ethylhexyl-1′-oxy)- | 88122-99-0 |
| | 1,3,5-triazine | |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: 5-Methyl-2-(1- | 134-09-8 |
| | methylethyl)-2-aminobenzoate | |
| 21 | Glyceryl p-aminobenzoate or: 1-Glyceryl 4- | 136-44-7 |
| | aminobenzoate | |
| 22 | 2,2′-Dihydroxy-4-methoxybenzophenone | 131-53-3 |
| | (dioxybenzone) | |
| 23 | 2-Hydroxy-4-methoxy-4-methylbenzophenone | 1641-17-4 |
| | (mexonone) | |
| 24 | Triethanolamine salicylate | 2174-16-5 |
| 25 | Dimethoxyphenylglyoxalic acid or: 3,4- | 4732-70-1 |
| | dimethoxyphenylglyoxalacidic sodium | |
| 26 | 3-(4′Sulfo)benzylidenebornan-2-one and its salts | 56039-58-8 |
| 27 | 4-tert-butyl-4′-methoxydibenzoylmethane | 70356-09-1 |
| 28 | 2,2′,4,4′-Tetrahydroxybenzophenone | 131-55-5 |
| 29 | 2,2′-Methylenebis[6(2H-benzotriazol-2-yl)-4- | 103597-45-1 |
| | (1,1,3,3,-tetramethyl-butyl)phenol] | |
| 30 | 2,2′-(1,4-Phenylene)bis-1H-benzimidazole-4,6- | 180898-37-7 |
| | disulfonic acid, Na salt | |
| 31 | 2,4-bis[4-(2-Ethylhexyloxy)-2-hydroxy]phenyl- | 187393-00-6 |
| | 6-(4-methoxy-phenyl)-(1,3,5)-triazine | |

Furthermore, the cosmetic and dermatological preparations according to the invention can advantageously comprise inorganic pigments based on metal oxides and/or other metal compounds which are insoluble or sparingly soluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. Particular preference is given to pigments based on TiO₂ and ZnO.

For the purposes of the present invention, it is particularly advantageous, although not obligatory, if the inorganic pigments are present in hydrophobic form, i.e. are superficially treated to repel water. This surface treatment can consist in providing the pigments with a thin hydrophobic layer in a manner known per se, as described in DE-A-33 14 742.

To protect human hair against UV rays, the mixtures according to the invention can be incorporated into shampoos, lotions, gels, hair sprays, aerosol foam creams, conditioners or emulsions in concentrations of from 0.1 to 10% by weight, preferably 1 to 7% by weight. The respective formulations can here be used, inter alia, for the washing, coloring and styling of hair.

The mixtures to be used according to the invention are generally notable for a particularly high absorption capacity in the region of UV-A and UV-B radiation with a sharp band structure. In addition, they are readily soluble in cosmetic oils and can be incorporated easily into cosmetic formulations. The emulsions prepared with the compounds I are notable in particular for their high stability, the compounds I themselves for their high photostability, and the preparations produced with I for their pleasant feel on the skin.

The UV filter effect of the mixtures according to the invention can also be utilized for stabilizing active ingredients and auxiliaries in cosmetic and pharmaceutical formulations.

In the examples below the preparation and use of the mixture is explained in more detail.

### EXAMPLES

Preparation

### Example 1

Preparation of a mixture of 40% by weight of n-hexyl 2-(4-diethylamino-2-hydroxybenzoyl)benzoate and 60% by weight of 2-ethylhexyl p-methoxycinnamate

400 g of the melt of n-hexyl 2-(4-diethylamino-2-hydroxybenzoyl)benzoate obtained according to DE-A-10221805 example 3 are admixed at room temperature with 600 g of 2-ethylhexyl p-methoxycinnamate and homogenized using a mechanical stirrer. The viscosity of this mixture at room temperature was 370 mPa*s (measured using Brookfield viscometer at 20 rpm).

General Procedure for Producing Emulsions for Cosmetic Purposes

All of the oil-soluble constituents are heated to 85° C. in a stirred tank. When all of the constituents have melted, or are in the form of a liquid phase, the water phase is incorporated with homogenization. With stirring, the emulsion is cooled to about 40° C., perfumed, homogenized and then cooled to 25° C. with continuous stirring.

Preparations

### Example 2

### Composition for Lip Care

Mass content (% by wt.)

| ad 100 | Eucerinum anhydricum |
|---|---|
| 10.00 | Glycerol |
| 10.00 | Titanium dioxide, micronized |
| 5.00 | Mixture from example 1 |
| 5.00 | Zinc oxide |
| 4.00 | Castor oil |
| 4.00 | Pentaerythrityl stearate/caprate/caprylate adipate |
| 3.00 | Glyceryl stearate SE |
| 2.00 | Beeswax |
| 2.00 | Microcrystalline wax |
| 2.00 | Quaternium-18 bentonite |
| 1.50 | PEG-45/dodecyl glycol copolymer |

### Example 3

### Composition for Sunblock with Micropigments

Mass content (% by wt.)

| ad 100 | Water |
|---|---|
| 10.00 | Octyl methoxycinnamate |
| 6.00 | PEG-7 hydrogenated castor oil |
| 6.00 | Titanium dioxide, micronized |
| 5.00 | Mixture from example 1 |
| 5.00 | Mineral oil |
| 5.00 | Isoamyl p-methoxycinnamate |
| 5.00 | Propylene glycol |
| 3.00 | Jojoba oil |
| 3.00 | 4-Methylbenzylidenecamphor |
| 2.00 | PEG-45/dodecyl glycol copolymer |
| 1.00 | Dimethicone |
| 0.50 | PEG-40 hydrogenated castor oil |
| 0.50 | Tocopheryl acetate |
| 0.50 | Phenoxyethanol |
| 0.20 | EDTA |

### Example 4

### Grease-Free Gel

Mass content (% by wt.)

| ad 100 | Water |
|---|---|
| 7.00 | Titanium dioxide, micronized |
| 5.00 | Mixture from example 1 |
| 5.00 | Glycerol |
| 5.00 | PEG-25 PABA |
| 1.00 | 4-Methylbenzylidenecamphor |
| 0.40 | Acrylate C10-C30 alkyl acrylate crosspolymer |
| 0.30 | Imidazolidinylurea |
| 0.25 | Hydroxyethylcellulose |
| 0.25 | Sodium methylparaben |
| 0.20 | Disodium EDTA |
| 0.15 | Fragrance |
| 0.15 | Sodium propylparaben |
| 0.10 | Sodium hydroxide |

### Example 5

### Sun Cream (SPF 20)

Mass content (% by wt.)

| ad 100 | Water |
|---|---|
| 8.00 | Titanium dioxide, micronized |
| 6.00 | PEG-7 hydrogenated castor oil |
| 13.00 | Mixture from example 1 |
| 6.00 | Mineral oil |
| 5.00 | Zinc oxide |
| 5.00 | Isopropyl palmitate |
| 0.30 | Imidazolidinylurea |
| 3.00 | Jojoba oil |
| 2.00 | PEG-45/dodecyl glycol copolymer |
| 1.00 | 4-Methylbenzylidenecamphor |
| 0.60 | Magnesium stearate |
| 0.50 | Tocopheryl acetate |
| 0.25 | Methylparaben |
| 0.20 | Disodium EDTA |
| 0.15 | Propylparaben |

### Example 6

### Sun Cream Water-Resistant

Mass content (% by wt.)

| ad 100 | Water |
|---|---|
| 5.00 | PEG-7 hydrogenated castor oil |
| 5.00 | Propylene glycol |
| 4.00 | Isopropyl palmitate |
| 4.00 | Caprylic/capric triglyceride |
| 13.00 | Mixture from example 1 |
| 4.00 | Glycerol |
| 3.00 | Jojoba oil |
| 2.00 | 4-Methylbenzylidenecamphor |
| 2.00 | Titanium dioxide, micronized |
| 1.50 | PEG-45/dodecyl glycol copolymer |
| 1.50 | Dimethicone |
| 0.70 | Magnesium sulfate |
| 0.50 | Magnesium stearate |
| 0.15 | Fragrance |

### Example 7

### Sun Milk (SPF 6)

Mass content (% by wt.)

| ad 100 | Water |
|---|---|
| 10.00 | Mineral oil |
| 6.00 | PEG-7 hydrogenated castor oil |
| 5.00 | Isopropyl palmitate |
| 8.00 | Mixture from example 1 |
| 3.00 | Caprylic/capric triglyceride |
| 3.00 | Jojoba oil |
| 2.00 | PEG-45/dodecyl glycol copolymer |
| 0.70 | Magnesium sulfate |
| 0.60 | Tocopheryl acetate |
| 3.00 | Glycerol |
| 0.25 | Methylparaben |
| 0.15 | Propylparaben |
| 0.05 | Tocopherol |

1. A mixture consisting of a UV-A filter of the formula I and a UV-B filter of the formula II.

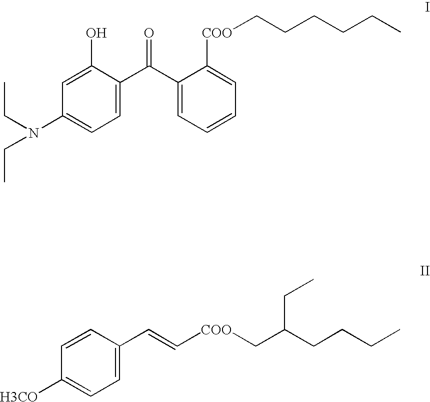

2. The mixture according to claim 1, consisting of 30 to 70% by weight of the UV-A filter I and 70 to 30% by weight of the UV-B filter II.

3. The mixture according to claim 1, in liquid form.

4. (canceled)

5. The mixture according to claim 2 in liquid form.

6. The mixture according to claim 1, consisting of 30 to 50% by weight of the UV-A filter I and 70 to 50% by weight of the UV-B filter II.

7. The mixture according to claim 1, consisting of 35 to 45% by weight of the UV-A filter I and 65 to 55% by weight of the UV-B filter II.

8. The mixture according to claim 1, wherein the mixture has a viscosity in the range from 100 to 800 mPa*s.

9. The mixture according to claim 7, wherein the mixture has a viscosity in the range from 200 to 600 mPa*s.

10. The mixture according to claim 1, wherein the mixture has a viscosity in the range from 300 to 500 mPa*s.

11. The mixture according to claim 6 in liquid form.

12. The mixture according to claim 7 in liquid form.

13. The mixture according to claim 9 in liquid form.

14. A cosmetic for protecting human skin and human hair against UV radiation. which comprises the mixture as claimed in claim 1.

15. A pharmaceutical for protecting human skin and human hair against UV radiation. which comprises the mixture as claimed in claim 1.

16. A cosmetic for protecting human skin and human hair against UV radiation. which comprises the mixture as claimed in claim 13.

17. A pharmaceutical for protecting human skin and human hair against UV radiation. which comprises the mixture as claimed in claim 13.

18. The cosmetic as claimed in claim 16, wherein the cosmetic is a composition for sunblock, grease free gel, sun cream, sun milk or sunscreen.
